(19) United States
(12) Patent Application Publication
(10) Pub. No.: US 2025/0339078 A1
(43) Pub. Date: Nov. 6, 2025

(54) ELECTRODE CONTACT MEASUREMENTS IN A MULTI-ELECTRODE SENSING SYSTEM

(71) Applicant: Apple Inc., Cupertino, CA (US)

(72) Inventors: Tao Shui, Cupertino, CA (US); Na Huang, Cupertino, CA (US)

(21) Appl. No.: 19/069,103

(22) Filed: Mar. 3, 2025

Related U.S. Application Data
(60) Provisional application No. 63/641,803, filed on May 2, 2024.

(51) Int. Cl. A61B 5/321; A61B 5/00

(52) U.S. Cl. CPC A61B 5/321; A61B 5/7228

(57) ABSTRACT

Embodiments are directed to systems and methods for determining a metric associated with electrode-skin contact quality. A reference signal is applied to a set of electrodes. The quality of contact between each electrode or subset of electrodes and the skin determines the amplitude of a contact signal that may be measured by processing circuitry of an electronic device. The contact signals for each of the electrodes, and any biological and/or other signals that may be acquired by the electrodes, are measured sequentially in a series of sampling windows. The measured signals associated with each sampling window are digitized and sequentially stored. A composite signal is generated from the sequentially stored samples and demodulated, to provide an output signal. The output signal may be analyzed to determine a metric associated with the contact quality between the set of electrodes and the skin of the user.

### CROSS-REFERENCE TO RELATED APPLICATIONS

This application is a nonprovisional and claims the benefit under 35 U.S.C. § 119(e) of U.S. Provisional Patent Application No. 63/641,803, filed May 2, 2024, the contents of which are incorporated herein by reference as if fully disclosed herein.

### FIELD

The described embodiments relate generally to determining metrics associated with electrode-skin contact quality. More specifically, the described embodiments are configured to apply a signal to a set of electrodes of an electronic device and generate a composite signal that may be used to determine electrode-skin contact.

### BACKGROUND

Many electronic devices may include electrodes that are used to acquire biological signals from a user. As an example, a smartwatch may include a set of electrodes used to measure an electrocardiogramaignal or other type of biological signal from the user. Measuring such a biological signal may require a baseline level of contact between the skin of the user and two or more of the device electrodes, and as such, an electronic device may use information about the level of skin-electrode contact in determining whether to perform a measurement and/or in analyzing a measurement. In some situations, the user may wish to begin a measurement but may not be in sufficient contact with one or more of the electrodes. In other situations, a measurement may be in progress but contact between the skin and one or more electrodes may become degraded or may be lost. Insufficient contact with one or more electrodes may result in poor signal quality and may affect aspects of the measurement.

### SUMMARY

Embodiments described herein are directed to systems and methods for determining a metric associated with the contact quality of a set of electrodes of an electronic device. Some embodiments are directed to a method for determining the metric by applying a reference signal to a set of electrodes. The method further includes sequentially measuring different subsets of the set of electrodes during a series of sampling windows and generating a series of samples for each of the sampling windows. The series of samples corresponding to each of the series of sampling windows are sequentially stored, and based on a sequence of the stored series of samples, a composite signal is generated. A representation of the reference signal is separated from the composite signal to form an output signal. The metric is determined based on the output signal.

In some instances, the method includes separating the representation of the reference signal from the composite signal by performing demodulation of the composite signal. In further instances, the demodulation comprises performing I/Q demodulation. In some examples, the demodulation may be performed using the reference signal as a demodulation signal.

In some variations, the method includes simultaneously applying the reference signal to each subset of the set of electrodes, or to each subset of electrodes. In other variations, the method includes sequentially applying the reference signal to each electrode of the set of electrodes, or to each subset of electrodes.

In some instances, the reference signal is applied to the subsets of the set of electrodes through a set of coupling capacitors. In some variations the reference signal is a sinusoid having a reference frequency. In additional variations, each series of samples is generated for each of the sampling windows at a measurement frequency that is an integer multiple of the reference frequency. In some instances, the measurement frequency is a multiple of two of the reference frequency.

Embodiments are also directed to a device that includes a set of electrodes, a signal generator configured to apply a first signal having a first frequency to each of a plurality of subsets of the set of electrodes, and processing circuitry. The processing circuitry is configured to sequentially measure each subset of the plurality of subsets of the set of electrodes to obtain a plurality of acquired signals, wherein each of the plurality of the acquired signals includes the first signal and a corresponding biological signal measured by a corresponding subset of the plurality of subsets of the set of electrodes. The processing circuitry is further configured to digitize the plurality of acquired signals and generate a composite signal using the digitized plurality of acquired signals. The processing circuitry demodulates the composite signal to separate the first signal from the corresponding biological signals of the digitized plurality of acquired signals. In some variations, the processing circuitry demodulates the composite signal using I/Q demodulation. The processing circuitry determines a metric associated with a contact quality of the set of electrodes using the demodulated composite signal.

In some variations, the device comprises a set of coupling capacitors, wherein the first signal is coupled by the set of capacitors to each of the plurality of subsets of set of electrodes. In further variations, the first signal is a sinusoid. In additional variations, the plurality of subsets of the set of electrodes is measured at a measurement frequency that is an integer multiple of the reference frequency. In still other variations the measurement frequency is a multiple of two of the first frequency.

Embodiments are also directed to a device that includes a set of electrodes, a signal generator configured to simultaneously apply a reference signal to each of a plurality of subsets of the set of electrodes, and processing circuitry. The processing circuitry is configured to sequentially measure, during a series of sampling windows, each of a plurality of subsets of the set of electrodes to generate a series of samples for each of the corresponding sampling windows in the series of sampling windows. The device is further configured to sequentially store, for each of the series of sampling windows, the corresponding series of samples and generate, based on the sequentially stored series of samples for each of the sampling windows, a composite signal. The representation of the reference signal is separated from the composite signal to form an output signal. Based on the output signal, the device determines a metric associated with contact quality of the set of electrodes. In some variations, the device separates the representation of the reference signal from the composite signal by performing demodulation of the composite signal. In some examples, the metric associated with contact quality is an average of the output signal determined by the device.

In some examples, processing circuitry of the device is further configured to generate, for each of the plurality of subsets of the set of electrodes, a corresponding output signal. Each corresponding output signal represents a portion of a contact signal acquired during a corresponding sampling window for each of the plurality of subsets of the set of electrodes. Further, each corresponding output signal is formed by separating the portion of the contact signal from a plurality of acquired signals that includes at least the contact signal and a biological signal.

In addition to the example aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIGS. 1 depicts a block diagram of an example electronic device that can be used to determine a metric associated with electrode-skin contact.

FIGS. 2A-2B depict views of an example electronic device that can determine a metric associated with electrode-skin contact.

FIG. 3A-3B depict an example timing sequence for measuring signals from subsets of electrodes.

FIG. 4 depicts portions of example processing circuitry that can be used by a device to measure and process signals acquired from subsets of electrodes.

FIG. 5 depicts portions of example processing circuitry that can be used to measure and process signals acquired from subsets of electrodes.

FIG. 6 depicts an example method for determining a metric associated with contact quality of a set of electrodes.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

An increasing number of electronic devices include a plurality of electrodes that can be used to measure various types of biological signals from a user. For example, a variety of smartwatches include a set of electrodes for acquiring biological signals associated with ECG. However, electronic devices that include a set of electrodes may be capable of acquiring biological signals associated with a range of electrical activity. In non-limiting examples, the device may be capable of acquiring biological signals associated with electromyography (EMG), electroencephalography (EEG), and/or biological signals associated with other types of electrical activity in the body of the user.

In some examples, the device may analyze the ECG, EMG, and/or other signals to evaluate a health condition of the user (e.g., evaluate the ECG signal for heart conditions), perform a task or function (e.g., use the EMG signal to control a device feature), and/or for other purposes. Analysis of the biological signals may be dependent on the signal quality of the acquired biological signals. For example, biological signals that are noisy, include significant artifacts (e.g., motion artifact), or otherwise have poor signal quality may hamper analysis of the biological signals.

One aspect of the measurement of biological signals that may affect signal quality is the contact quality between one or more of the electrodes and the skin of the user. For example, poor contact between an electrode (or electrodes) and the skin may reduce the amplitude of biological signals, may increase noise, or may otherwise affect signal quality. Factors that may affect electrode-skin contact quality may include the contact surface area between the electrode and skin, the amount of force (or pressure) applied between the skin and electrode, the moisture content of the user's skin, foreign matter between the electrode and skin (e.g., solid or liquid foreign matter), and/or other factors.

In some examples, contact quality between the electrode and skin may vary over the course of a biological signal measurement or may be poor for the entirety of the measurement. For example, during a measurement, the posture of the user may shift, causing contact with one or more of the electrodes to degrade (e.g., contact area decreases) or to be lost entirely (e.g., complete loss of contact between the electrode and the skin). In other examples, the user may start a measurement while (knowingly or unknowingly) failing to make contact with one or more electrodes. For instance, the user may start an ECG measurement but may not establish contact, or may establish insufficient contact, between a finger and a corresponding electrode. Consequently, the signal quality of the acquired biological signals may be insufficient for analyzing the biological signals, or the biological signals may be completely absent, due to total lack of electrode-skin contact.

Embodiments disclosed herein are directed to systems and methods for performing electrode-skin contact measurements in a device that includes a set of electrodes used to acquire a biological signal (or signals) from a user. The set of electrodes includes electrodes intended for contact with a user's wrist, finger, and/or other portion of the user's body. Data acquired during a contact measurement may be used to determine one or more metrics that represent the quality of contact between the user and one or more of the electrodes. These metrics may be used to alter or control operation of the device.

In some examples, during a measurement, a reference signal is applied to the set of electrodes. The reference signal may be applied with a particular amplitude at each moment in time. The contact quality between each electrode and the skin may affect (e.g., reduce) the amplitude of the signal ultimately appearing at each electrode. For instance, the electrode-skin contact quality may affect the electrode-skin impedance of each electrode, which may further affect the amplitude of the signal (also referred to herein as a “contact signal”) appearing at each electrode or across a subset of electrodes. A corresponding contact signal may be measured from each electrode (or subset of electrodes) at different times during a measurement. The devices described herein are configured to measure and analyze the collective contact signals associated with the set of electrodes and determine one or more metrics of contact quality therefrom.

While the reference signal is applied, different subsets of the electrodes (e.g., each of a plurality of subsets of the electrodes) are sequentially measured during each of a series of sampling windows. Each subset of the plurality of subsets of electrodes includes two or more electrodes, as described herein. Measurements collected during each sampling window may be digitized, such as by an analog-to-digital converter (ADC) or other suitable component, to form a series of one or more samples for each sequential sampling window. A composite signal is generated by assembling the corresponding series of samples from each of the sampling windows in sequential order. The composite signal may include signals from several sources. For example, the composite signal may include a representation of the applied reference signal, biological signals (ECG, EMG, etc.), environmental signals, noise, measurement artifacts, and the like.

The representation of the reference signal is separated from the composite signal to form an output signal associated with electrode-skin contact. In some examples the representation of the reference signal may be separated from the composite signal using a signal demodulation method, such as IQ demodulation. In other examples, the representation of the reference signal may be separated from the composite signal using another method.

Based on the output signal, a metric associated with electrode-skin contact may be determined. For example, the metric may be an average of the output signal, where the average is computed over a time period of the output signal and reflects the average contact quality of multiple subsets of electrodes over that time period. In other examples, another type of metric may be affected by the quality of electrode-skin contact, and may be determined by the device. The metric may be compared to a predetermined threshold, or a predetermined range of values. The threshold (or range of values) may represent a suitable level of signal quality and if the metric exceeds the threshold (or is outside the predetermined range), the device may determine that the user is not making sufficient contact with one or more of the electrodes.

In some examples, the metric may be determined throughout the measurement. For instance, the metric may be determined beginning from the commencement of the measurement in order to determine when a threshold level of contact is made (e.g., by the wrist, finger, etc.) with the electrodes associated with the measurement. Determination of the metric may continue throughout the measurement, to ensure adequate electrode-skin contact during the entirety of the measurement. In instances when the device determines poor contact between the user and one or more of the electrodes, such as when the metric exceeds a threshold, the device may prevent the measurement from continuing (or from starting). In some instances, the device may allow the measurement to continue, but may advise or guide the user to improve electrode contact.

In other instances, if the device determines poor electrode-skin contact during a portion of the measurement, the device may disregard data samples collected during that portion of the measurement (e.g., during the time when contact is poor), may extend the measurement to collect additional samples, and/or may otherwise allow the measurement to continue.

In still other instances, the device may perform analysis of the signals acquired during the measurement only after the measurement is complete. If the device determines poor electrode contact during a portion (or the entirety) of the measurement, the device may proceed as described above. That is, the device may disregard samples during periods of poor contact, may disregard the entire measurement, and/or may take other action with regard to the completed measurement.

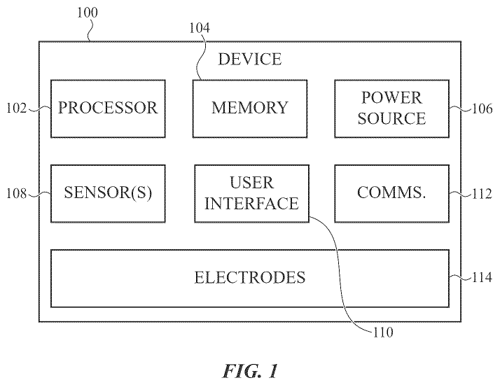

These and other embodiments are discussed below with reference to FIGS. 1-6. However, those skilled in the art will readily appreciate that the detailed description given herein with respect to these Figures is for explanatory purposes only and should not be construed as limiting.

FIG. 1 depicts a block diagram of an example electronic device 100 that can be used to determine a metric associated with contact quality for a set of electrodes 114. The device 100 can include a processor 102, memory 104, a power source 106, one or more sensors 108, a user interface 110, a communications unit 112, and the set of electrodes 114.

The processor 102 can control some or all of the operations of the device 100. The processor 102 can communicate, either directly or indirectly, with some or all of the components of the device 100. For example, a system bus or other communication mechanism can provide communication between the processor 102, the memory 104, the power source 106, the one or more sensors 108, the user interface 110, the communications unit 112, and elements associated with the electrodes 114.

The processor 102 can be implemented as any electronic device capable of processing, receiving, or transmitting data or instructions. For example, the processor 102 may include a processor, a microprocessor, a graphics processing unit (GPU), a programmable logic array (PLA), a programmable array logic (PAL), a generic array logic (GAL), a complex programmable logic device (CPLD), an application-specific integrated circuit (ASIC), a field-programmable gate array (FPGA), or any other programmable logic device (PLD) configurable to execute an operating system and applications of device 100, as well as to facilitate acquisition and processing of signals as described herein. The term “processor,” as used herein, is meant to encompass a single processor or processing unit, multiple processors, multiple processing units, or other suitable computing element or elements.

It should be noted that the components of the device 100 can be controlled by multiple processors. For example, select components of the device 100 (e.g., a sensor 108) may be controlled by a first processor and other components of the device 100 (e.g., the communications unit 112) may be controlled by a second processor, where the first and second processors may or may not be in communication with each other.

The memory 104 can store electronic data that can be used by the electronic device 100. For example, the memory 104 can store electrical data or content such as, for example, measured electrical signals, audio and video files, documents and applications, device settings and user preferences, timing signals, control signals, and data structures or databases. The memory 104 can include one or more non-transitory computer-readable storage devices, for storing computer-executable instructions, which, when executed by one or more computer processors 102, for example, can cause the computer processors to perform the techniques that are described herein. A computer-readable storage device can be any medium that can tangibly contain or store computer-executable instructions for use by or in connection with the instruction execution system, apparatus, or device. In some examples, the storage device is a transitory computer-readable storage medium. In some examples, the storage device is a non-transitory computer-readable storage medium. The non-transitory computer-readable storage device can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like.

The power source 106 can be implemented with any device capable of providing energy to the device 100. For example, the power source 106 may be one or more batteries or rechargeable batteries. The power source 106 may include battery charging components within the device 100, which may receive power, charge the battery, and/or provide direct power to operate the device 100 regardless of the battery's state of charge (e.g., bypassing the battery of the device 100). In some cases, the battery charging components may include a coil such that the device 100 may receive power wirelessly (e.g., via inductive power transfer). The device 100 may include a magnet, such as a permanent magnet, that magnetically couples to a magnet (e.g., a permanent magnet, electromagnet) or magnetic material (e.g., a ferromagnetic material such as iron, steel, or the like) in a charging dock (e.g., to facilitate wireless charging of the device 100).

The device 100 also includes one or more sensors 108. The sensor(s) 108 can be configured to sense one or more type of parameters, such as but not limited to, electrical signals, pressure, sound, light, touch, heat, movement, relative motion, biometric data (e.g., physiological parameters), and so on. For example, the sensor(s) 108 may include one or more pressure sensors, auditory sensors, heat sensors, position sensors, light or optical sensors, accelerometers, pressure transducers, gyroscopes, magnetometers, GPS sensors, health monitoring sensors, and so on. The health monitoring sensors may include an optical or other type of heart rate sensor, an ECG, an EMG, an EEG, and/or other types of health sensors. Additionally, the one or more sensors 108 can utilize any suitable sensing technology, including, but not limited to, capacitive, ultrasonic, resistive, optical, ultrasound, piezoelectric, and thermal sensing technology.

The sensor(s) 108 may further include analog and/or digital processing circuitry that may be associated with acquiring and/or processing signals associated with the above-described sensors and/or sensing technologies. In non-limiting examples, such processing circuitry may include amplifiers, signal filters, analog-to-digital converters (ADCs), memory elements, and/or other types of components or elements. The sensor(s) 108 may also include additional circuitry used to configure sensor measurements. For example, the sensor(s) 108 may include a set of one or more switches, multiplexers, signal generators, and/or other circuitry associated with acquiring sensor measurements. In a health sensor example, one or more circuit elements of the sensor(s) 108 may be coupled to one or more of the electrodes 114, such as with an ECG sensor, EMG sensor, EEG sensor, or the like. When one or more circuit elements of the sensor(s) 108 are coupled to one or more electrodes of the set of electrodes 114, those electrodes may be considered part of that sensor. In some variations, the set of electrodes 114 (or a portion thereof) may be part of different sensors at different times. For instance, a device may include two sensors (e.g., an ECG sensor and an EMG sensor) that use the same set or subset of electrodes at different times (e.g., the ECG sensor may use a set of electrodes to perform an ECG measurement at a first time and the EMG sensor may use the same set of electrodes at other times to perform an EMG measurement). Circuit elements associated with these sensors may also be associated with acquiring/processing a biological signal from the user, and/or with determining contact between the skin of the user and one or more of the electrodes 114, as described herein.

The device 100 includes a user interface 110, which may include a type of graphical display. The display may be implemented as a liquid-crystal display (LCD), organic light-emitting diode (OLED) display, light-emitting diode (LED) display, or the like. If the display is an LCD (or other type of display technology), the display may also include a backlight component that can be controlled to provide variable levels of display brightness. If the display is an OLED or LED type display (or other type of display technology), the brightness of the display may be controlled by modifying the electrical signals that are provided to display elements. In some examples, the display may be a type of touch-sensitive display (e.g., a capacitive touch display) that allows a user to provide input to the device 100 via touch-based interaction with the display screen.

The user interface 110 may include other types of user interface elements. For example, the user interface can include one or more buttons, dials, switches, knobs, levers, and/or other types of inputs. In some examples, the user interface 110 may include a type of rotatable input device or a depressible and rotatable input device (such as the rotatable and depressible crown associated with a smartwatch). In additional examples, the user interface 110 may include one or more cameras, one or more microphones, one or more speakers, a keyboard, and/or other types of user interface elements.

In further examples, the user interface 110 may provide graphical user interface (GUI) elements on the display of the device. For example, the user interface 110 may provide virtual buttons (e.g., a graphical user interface “home” button), slide controls, and/or any of a variety of other types of virtual user inputs and/or controls on the display of the device 100. The user interface 110 may further provide graphical output, such as text, lists, symbols, signals, waveforms, photographs, videos and/or other graphics, to the display of the device 100. In still further examples, the user interface 110 may provide a GUI to a display of the device 100, where one or more graphical objects of the GUI display information collected from or derived from one or more of the sensor(s) 108. For example, the user interface 110 may output information related to a measurement performed by a particular sensor, such as the type of measurement performed, the status of the measurement (e.g., “in progress” or “completed”), the results of the measurement, or so on.

The communications unit 112 can transmit data to, and/or receive data from, another electronic device. The communications unit 112 can transmit/receive electronic signals via a communications network, such as a wireless and/or wired network connection. Examples of wireless and wired network connections include, but are not limited to, NFC, RF, cellular, Wi-Fi, Bluetooth, IR, and Ethernet connections. The communications unit 112 may further include one or more ports and/or connectors for establishing wired connection with another device or devices.

The device 100 includes a set of one or more electrodes 114. The electrodes 114 may be positioned on an exterior, user-accessible surface of the device, such as on a surface of the device housing, on a surface of a user interface element (e.g., a button, a dial, etc.), and/or on other surfaces of the device 100. The electrodes 114 may be fabricated from any of a variety of suitable materials, which may provide a range of conductivity values. For example, the electrodes 114 may be a type of metal, glass, ceramic, composite, or other type of material. In some examples, all of the electrodes 114 may be the same type of material, while in other examples one subset of one or more of the electrodes 114 may be comprised of a different material than another subset or subsets of the electrodes 114.

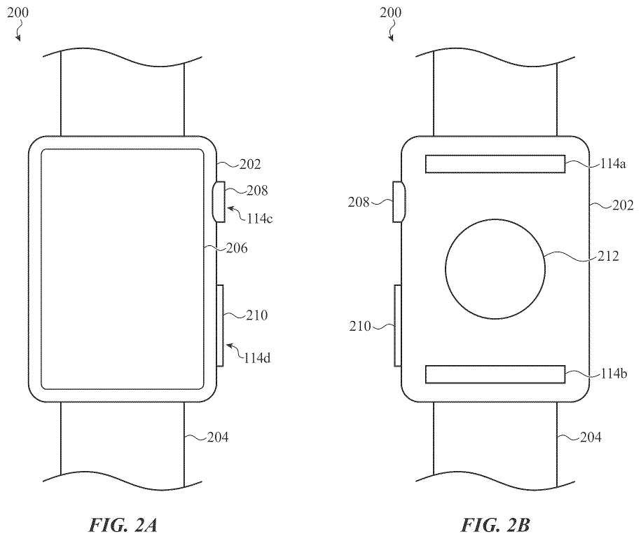

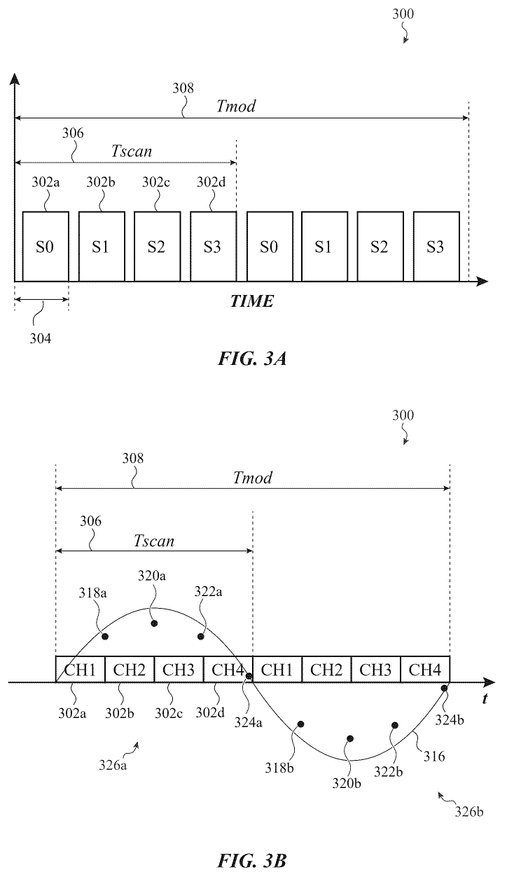

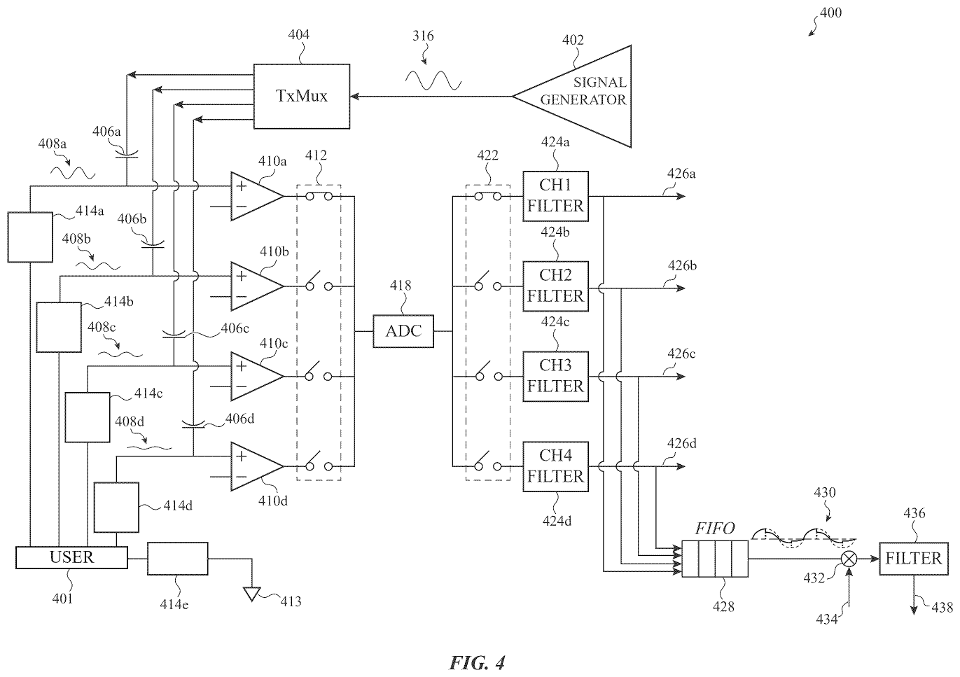

In general, the set of electrodes includes multiple electrodes. For instance, the device 100 may include three or more electrodes, such as depicted with respect to FIGS. 2A-2B and 4. Depending on the type of sensor and the measurement being performed, a given electrode of the set of electrodes may be configured as a sensing electrode or as a reference electrode. In some variations, a reference electrode may be used to establish connection between the user and a reference point of the associated sensor processing circuitry (e.g., a circuit ground or other reference point), and a sensing electrode may measure a potential relative to the reference point. In further examples, two or more electrodes may be grouped as a subset of electrodes and referred to as a channel, such as described herein with respect to FIGS. 3 and 4. A subset of electrodes may include, one or more sensing electrodes and one or more reference electrodes. In certain variations, the device 100 may not include a reference electrode, and each subset of electrodes may be comprised entirely of sensing electrodes.

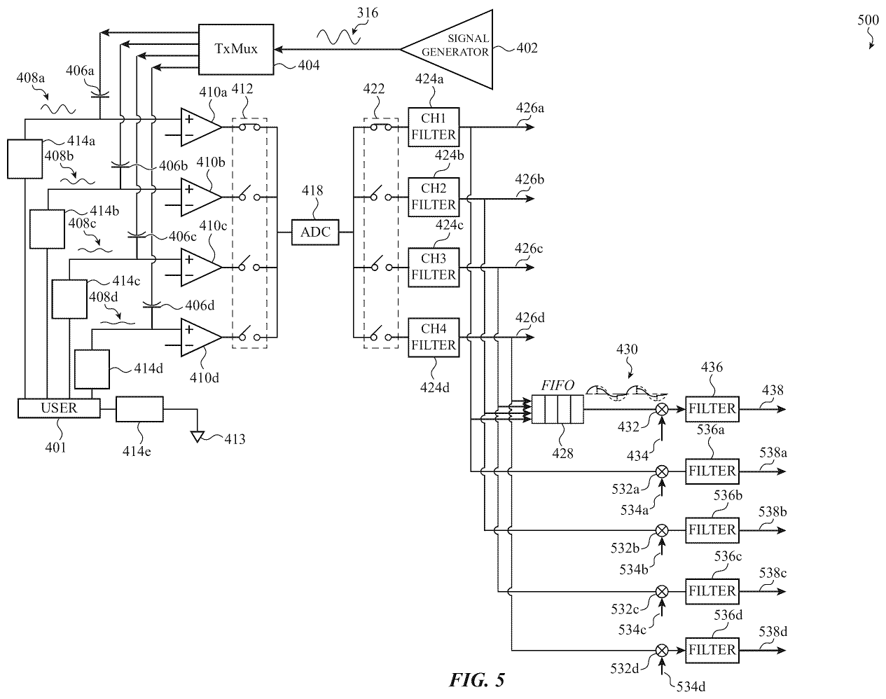

As described herein, processing circuitry and/or other circuitry associated with the acquisition and processing of signals acquired from one or more of the electrodes 114 may be part of processor 102, sensor(s) 108, electrodes 114, and/or other elements of device 100. Exemplary circuit elements of device 100 are depicted in FIGS. 4-5 and described herein.

FIG. 2A shows a front view of an example smartwatch 200 and FIG. 2B shows a back view of the example smartwatch 200 which can be used to determine a metric associated with electrode-skin contact, as described herein. The smartwatch 200 may include the elements described above with respect to device 100 of FIG. 1, and may be capable of performing any or all the functions of device 100 described herein. The smartwatch 200 is merely one example embodiment of an electronic device, and the concepts discussed herein may apply equally or by analogy to other electronic devices, including, smart band, mobile phones (e.g., smartphones), tablet computers, notebook computers, head-mounted display devices, headphones, earbuds, digital media players (e.g., mp3 players), or the like.

The smartwatch 200 includes a housing 202 and a band 204 coupled to the housing. The housing 202 may at least partially define an internal volume in which components of the smartwatch 200 may be positioned. The housing 202 may also define one or more exterior surfaces of the device, such as all or a portion of one or more side surfaces, a rear surface, a front surface, and the like. The housing 202 may be formed of any suitable material, such as metal (e.g., aluminum, steel, titanium, or the like), ceramic, polymer, glass, or the like. The band 204 may attach the smartwatch 200 to a user, such as to the user's arm, wrist, or other portion of the user's body.

The smartwatch 200 further includes user interface elements, such as described herein for user interface 110 of FIG. 1. For example, the smartwatch 200 includes display 206, a first input device 208, and a second input device 210. The display 206 may be configured in any manner as described herein. For instance, the display 206 may be a type of touch-sensitive display capable of receiving input via touch interaction from the user. The smartwatch 200 may display the output of sensor measurements on the display 206, such as data or information associated with a measurement performed by a sensor, as described herein (e.g., an ECG measurement, EMG measurement, electrode-skin contact measurement, etc.).

The first input device 208 may have a cap, crown, protruding portion, or component(s) or feature(s) positioned along a side surface of the housing 202. At least a portion of the first input device 208 (such as a crown body) may protrude from, or otherwise be located outside, the housing 202, and may define a generally circular shape or circular exterior surface. The exterior surface of the first input device 208 may be textured, knurled, grooved, or otherwise have features that may improve the tactile feel of the first input device 208 and/or facilitate rotation sensing.

The first input device 208 may facilitate a variety of potential interactions. For example, the first input device 208 may be rotated by a user (e.g., the crown may receive rotational inputs). Rotational inputs of the first input device 208 may zoom, scroll, rotate, or otherwise manipulate a user interface or other object displayed on the display 206 among other possible functions. The first input device 208 may also be translated or pressed (e.g., axially) by the user. Translational or axial inputs may select highlighted objects or icons, cause a user interface to return to a previous menu or display, or activate or deactivate functions among other possible functions.

In some cases, the smartwatch 200 may sense touch inputs or gestures applied to the first input device 208, such as a finger sliding along the body of the first input device 208 (which may occur when first input device 208 is configured to not rotate) or a finger touching the body of the first input device 208. In such cases, sliding gestures may cause operations similar to the rotational inputs, and touches on a cap or crown may cause operations similar to the translational inputs. As used herein, rotational inputs include both rotational movements of the first input device 208, as well as sliding inputs that are produced when a user slides a finger or object along the surface of a crown in a manner that resembles a rotation (e.g., where the crown is fixed and/or does not freely rotate).

The smartwatch 200 may also include other input devices, switches, buttons, or the like. For example, the smartwatch 200 includes a second input device 210, which may be a button. The second input device 210 may be a movable button or a touch-sensitive region of the housing 202. The button may control various aspects of the smartwatch 200. For example, the button may be used to select icons, items, or other objects displayed on the display 206, to activate or deactivate functions (e.g., to silence an alarm or alert), or the like.

FIG. 2B shows a rear side of the smartwatch 200. The smartwatch 200 may include one or more windows 212 (one of which is shown), which may be coupled to the housing 202 and which may allow light to pass through a portion of the housing 202. The one or more windows 212 may be part of an optical sensing system, which may further be incorporated within a health sensor or other type of sensor (e.g., sensor(s) 108). The one or more windows 212 may include light transmissive materials and be associated with internal sensor components, which may be used to determine biometric information of a user, such as heart rate, blood oxygen concentrations, and the like, as well as information such as a distance from the smartwatch to an object. The particular arrangement of the one or more window(s) 212 in the housing 202 shown in FIG. 2B is one example arrangement, and other window arrangements (including different numbers, sizes, shapes, and/or positions of the windows) are also contemplated. As described herein, the window arrangement may be defined by or otherwise correspond to the arrangement of components in the integrated sensor package.

The smartwatch 200 is depicted in FIGS. 2A and 2B as including a set of four electrodes 114a-114d (e.g., a first electrode 114a, a second electrode 114b, a third electrode 114c, and a fourth electrode 114d) for acquiring a biological signal from the user. In other examples, the smartwatch (or other device) may include more or fewer electrodes. The electrodes of the set of electrodes 114a-114d may be positioned on any portions of the smartwatch 200 as may be needed to perform measurements using the sensors described herein.

For example, a rear-facing portion of the housing 202 (e.g., a surface of the housing 202 opposite the display 206) may include one or more electrodes. In the example depicted in FIG. 2B, a rear-facing surface of the smartwatch 200 includes two electrodes of the set of electrodes 114a-114b (e.g., the first electrode 114a and the second electrode 114b). In some examples, electrodes 114a-114b may be used to make contact with the user's wrist, other portion of the user's arm, or other portion of the user's body. In some examples, electrodes 114a-114b may include more or fewer electrodes than depicted in FIG. 2B. Further, the electrodes 114a-114b may be located on other portions of the housing 202, may be include electrodes with different shapes than depicted, and/or may be positioned and/or oriented on the housing 202 according to another arrangement.

In the example depicted in FIG. 2A, the smartwatch 200 may include additional electrodes of the set of electrodes (e.g., the third electrode 114c and fourth electrode 114d) for making contact with the finger of the user, or with another portion of the user's body. For example, a surface of first input device 208 and/or second input device 210 may include electrodes 114c-114d, respectively. In some variations, the surface of the first input device 208 may include a single electrode (such as depicted), two electrodes, three electrodes, or more. Similarly, the surface of the second input device 210 may include single electrode (such as depicted), two electrodes, three electrodes, or more.

The electrodes 114a-114d may be conductively coupled to processing and/or other circuitry within the housing 202, such as described herein (e.g., associated with processor 102, sensor(s) 108, and/or the electrodes 114a-114d themselves). Example circuitry is described herein with respect to FIGS. 4-5. This circuitry may be associated with determining a metric associated with contact between the skin of the user and the electrodes 114a-114d.

When a sensor of the electronic devices described herein perform a measurement using a set of electrodes, the sensor may be configured to sequentially measure different subsets of the set of electrodes. FIGS. 3A-3B depict an example timing sequence 300 that indicates how an electronic device obtains measurements from different electrodes, or subsets of electrodes, over time. The timing sequence 300 may be implemented by a device, such as example devices 100 and 200. The timing sequence 300 includes a series of sequential sampling windows 302a-302d, which correspond, respectively, to a first sampling window S0, a second sampling window S1, a third sampling window S2, and a fourth sampling window S3. During each sampling window 302a-302d, the device measures the signal present between a corresponding subset of two or more electrodes. Accordingly, for each series of sampling windows 302a-302d, a plurality of subsets of the set of electrodes will be measured. For ease of discussion, each subset of electrodes may be referred to herein as a “channel” or as an “electrode channel,” such as indicated in FIG. 3B. In the example depicted, a first channel corresponding to a first subset of electrodes is measured during the first sampling window 302a, a second channel corresponding to a second subset of electrodes is measured during the second sampling window 302b, and so on. Thus, FIGS. 3A-B depict an example where a device is configured to provide sequential measurement of four electrode channels. In some examples, the timing sequence 300 may include greater or fewer sampling windows 302a-302d, corresponding to greater or fewer configured electrode channels.

Each electrode channel may correspond to a unique subset of two or more electrodes (e.g., one or more of the electrodes may be configured as sensing electrodes, and one or more electrodes may be configured as reference electrodes). For instance, CH1 may be configured to include a first electrode and a second electrode; CH2 may be configured to include a third electrode and a fourth electrode; and so on. In other examples, each electrode channel may include a set of one or more electrodes that are common between the channels. For example, CH1 may include a first electrode, CH2 may include a second electrode, and both CH1 and CH2 may include a third electrode. In some of these examples, the third electrode may be a reference electrode, such as described herein, that is shared between CH1 and CH2.

In still other examples, each channel may be configured with three or more electrodes, some of which may be unique to each channel, while others may be common to one or more of the channels (e.g., one or more reference electrodes). An example electrode configuration that includes a common reference electrode used with all electrode channels is depicted in FIGS. 4-5. Other examples are possible.

During each of the sampling windows 302a-302d, the device performs one or more signal measurements from the channel corresponding to that sampling window. During each measurement, the device acquires a signal from the corresponding subset of electrodes (e.g., the corresponding electrode channel). This acquired signal corresponds to a measured electrical potential between the electrodes of the subset of electrodes that form the channel. The acquired signal may be further processed (e.g., amplified and/or filtered) and digitized to generate one or more samples from the acquired signal. In some instances, the signal acquired during each sampling window 302a-302d may also be processed during each sampling window. In other instances, the acquired signal may be processed following the conclusion of the measurement, or at other times as may be desired. The data samples generated during each sampling window 302a-302d may be sequentially combined to generate a composite signal, as described herein with respect to FIGS. 4-5. In this regard, the composite signal may contain samples collected from each of the different subsets of electrodes.

Each of the sampling windows 302a-302d may be configured to occur for the same measurement duration 304, which may include a setup period that may occur before and/or after each of the sampling windows 302a-302d. The setup period may provide time for the device to transition from measurement of one electrode channel to another, such as may be needed to actuate one or more switches, allow for settling time, and/or for other purposes. In some examples, no setup time may be required between each sampling window 302a-302d, such that each sampling window 302a-302d may occupy (or substantially occupy) the entire measurement duration 304. In some examples, one or more of the sampling windows 302a-302d may each have the same measurement duration 304, while the remaining sampling windows 302a-302d may have, or may each have, a different measurement duration 304. In still other examples, all of the sampling windows 302a-302d may have a different measurement duration 304 from one another.

In the example depicted in FIGS. 3A-B, measurements are acquired from each electrode channel sequentially, during a measurement period 306. As used herein, a “measurement period” refers to a period of time that includes each of the series of sampling windows 302a-302d. A complete measurement period 306 that includes all four sampling windows 302a-302d (for each of the configured electrode channels) lasts for a duration of Tscan, and has a corresponding measurement frequency (Fscan, where Fscan=1/Tscan). In timing sequence 300, the measurement period 306 is complete at the end of the fourth sampling window (S3) 302d, with the sampling windows 302a-302d occupying the full measurement period 306, or a substantial portion of the measurement period 306. For instance, the sampling windows 302a-302d may occupy more than 85%, more than 90%, or more than 95% of the measurement period 306. In other example timing arrangements, the sampling windows 302a-d may occupy a smaller portion of the measurement period 306, such as less than 85%, less than 80%, or less than 75% of the measurement period 306.

In addition, a measurement may include multiple measurement periods 306 during which the sequence of sampling windows 302a-302d (and their corresponding measurements) are repeated. Two such measurement periods are depicted in FIGS. 3A-B. In other examples, the measurement may include additional measurement periods 306 that repeat over the entirety of the measurement. In some variations, the measurement sequence may change between successive measurement periods 306 (e.g., certain subsets of electrodes may be added or removed, or the relative time spent in each measurement window may change). Further, the order in which each channel is measured may also be modified from an initial order.

During a measurement, the device also generates a reference signal 316 that is applied to certain electrodes during the measurement. Specifically, during each sampling window 302a-302d, the reference signal 316 is applied to at least the subset of electrodes that is being measured during that sampling window. In some variations, the reference signal 316 is also applied to electrodes that are not currently being measured during a given sampling window 302a-302d. The reference signal 316 may be generated by a signal generator of the device. FIGS. 4-5 depict an example of how the reference signal 316 may be generated and coupled to the set of electrodes.

The reference signal 316 may be a repeating signal that has a reference period (Tmod) 308, and a corresponding reference frequency (Fmod, where Fmod=1/Tmod). In the example depicted, the reference period 308 is twice the measurement period 306 (i.e., the reference frequency Fmod is half the measurement frequency Fscan), but this relationship is not required. That is, the reference period 308 of the reference signal 316 is not required to be an integer multiple of the measurement period 306. The reference signal 316 may have a reference period 308 of any duration suitable for determining electrode-skin contact as described herein. Further, the reference signal 316 may be sinusoidal (as depicted) or may be another type of signal suitable for determining electrode-skin contact. For example, the reference signal 316 may be a triangle wave or other type of periodic signal.

As described herein, during a measurement, with the reference signal 316 applied to the set of electrodes, a series of one or more samples are generated during each of the sampling windows 302a-302d. In the example depicted, a single sample is generated during each sampling window 302a-302d. In other examples, additional samples may be generated during each of the sampling windows 320a-302d.

During one cycle of the reference signal 316 (e.g., reference period 308), two samples are generated for each electrode channel across two different corresponding sampling windows. Samples 318a-b are generated for channel 1, samples 320a-320b are generated for channel 2, and so on. Using channel 1 as an example, during the sampling window 302a, sample 318a is generated during a first phase 326a (e.g., a positive phase) of the reference signal 316. Sample 318a is at least partially comprised of portions of the contact signal, which as described herein, may have a lower amplitude than the reference signal 316. Sample 318a may further be comprised of biological and/or other signals acquired by a subset of one or more electrodes associated with channel 1. Similarly, a subsequent sample of channel 1 (sample 318b) is generated during a second phase 326b (e.g., a negative phase) of the reference signal 316, and may be similarly comprised of the same signals as sample 318a. The remaining samples, 320a-320b, 322a-322b, and 324a-324b, may be comprised of the same or similar signals, and are generated for the respective samplings windows indicated in FIG. 3B. These samples may be analyzed to determine a metric associated with the contact quality, such as described herein.

FIG. 4 depicts portions of example processing circuity 400 that may be included in a device and used to measure and process signals acquired from a set of electrodes 414a-414e. In the example depicted, the processing circuitry 400 is configured to acquire signals from four subsets of the electrodes 414a-414e, where each subset of electrodes represent an “electrode channel.” Each of the electrode channels is comprised of one of the sensing electrodes 414a-414d, respectively, and a common reference electrode 414e. The reference electrode 414e provides connection between the user 401 and the circuit reference (or circuit ground) 413.

As described herein, the electrodes 414a-414e may be arranged in different locations on an exterior surface of the device, such that the electrodes 414a-414e may make contact with different parts of body of the user 401. In examples where the device is a wearable device, such as the smartwatch 200 depicted in FIGS. 2A-B, a first subset of the electrodes 414a-414e may be positioned so as to contact the wrist of the user 401 and a second subset of the electrodes 414a-414e may be positioned to be accessible to a finger of the user 401. For example, the first electrode 414a may be positioned so that the user 401 may contact the first electrode 414a with a finger and the remaining electrodes 414b-414e may be positioned to contact the wrist of the user 401, when the device is worn.

As described herein, an impedance exists between each of the electrodes 414a-414e and the user 401. For example, the impedance represented by of each electrode 414a-414e may be based on surface area of contact between each electrode 414a-414e and the skin of the user 401 (or a complete lack of contact), the presence of moisture between each electrode 414a-414e and the user 401, the skin condition of the user 401, the type of skin in contact with each electrode 414a-414e (e.g., glabrous or non-glabrous skin), and/or a range of other factors.

The processing circuitry 400 includes a signal generator 402 that generates reference signal 316. The signal generator 402 may be a type of digital-to-analog converter (DAC), or may be another type of component or circuit capable of generating a suitable reference signal 316. As described above, the reference signal 316 is depicted as a sinusoid, but may be any suitable reference signal as described herein.

The signal generator 402 may provide the reference signal 316 to a multiplexer 404, which distributes the reference signal 316 to each of the electrodes 414a-414d. The multiplexer 404 may provide the reference signal 316 to each of the electrodes 414a-414d according to a predetermined sequence (e.g., one at a time, in a predefined order), or may provide the reference signal 316 to all of the electrodes 414a-414d simultaneously.

In the example depicted, a multiplexer 404 is used to distribute the reference signal 316. However, in other examples, the multiplexer 404 may be implemented as a set of switches or other type of component(s) suitable for distributing the reference signal 316 to the electrodes 414a-414d.

The reference signal 316 may be coupled to the sensing electrodes 414a-414d via a set of coupling capacitors 406a-406d, whereby each electrode of the electrodes 414a-414d is respectively connected to a coupling capacitor of the set of coupling capacitors 406a-406d. The coupling capacitors 406a-406d couple the reference signal 316 to any signals that are acquired by each of the electrodes 406a-406d (e.g., biological signals, measurement artifact signals, etc.), such that the reference signal 316 is combined with these signals for further processing.

As described herein, the electrodes 414a-414e are grouped into four subsets (e.g., channels), where each subset includes one of the sensing electrodes 414a-414d and the (shared) reference electrode 414e. In other examples (not depicted in FIG. 4), the device may include one or more subsets of electrodes that include two or more sensing electrodes and a reference electrode (e.g., the reference electrode 414e). In such examples, the reference signal 316 may be coupled to each of the sensing electrodes of each subset of electrodes via separate coupling capacitors (e.g., one coupling capacitor connected to each sensing electrode of the subset). In some variations, the reference signal 316 may be coupled to all sensing electrodes of a subset of electrodes via a single coupling capacitor.

In some examples, the coupling capacitors 406a-406d may have identical capacitance values. In other examples, one or more of the coupling capacitors 406a-406d may have different values than one another. The impedance of the coupling capacitors 406a-406d depends on the capacitance value of each capacitor 406a-406d and the frequency of the reference signal 316.

The impedance of the electrodes 414a-414e also depends on the frequency of the reference signal 316, in addition to the other factors described herein (e.g., type of skin, moisture content of the skin, contact surface area, etc.). The impedance of the reference electrode 414e combined with the impedance of each electrode 414a-414d forms a voltage divider with the impedance of each of the respective coupling capacitors 406a-406d. This voltage divider reduces the amplitude of the applied reference signal 316, based on the combined electrode impedance, resulting in a reference signal representation (e.g., contact signals) 408a-408d for each electrode channel. Higher combined electrode impedances result in higher amplitude contact signals 408a-408d, and conversely, lower combined electrode impedances result in lower amplitude contact signals 408a-408d.

Using channel 1 as an example, the first electrode 414a exhibits a first impedance at the frequency of the reference signal 316 based on the quality of contact between the user 401 and the first electrode 414a. The reference electrode 414e exhibits a second impedance at the frequency of the reference signal 316 based on the quality of contact between the user 401 and reference electrode 414e. The combination of the first and second impedances forms a voltage divider with the first coupling capacitor 406a, which exhibits a (fixed) third impedance at the frequency of the reference signal 316 that is not based on electrode contact quality. In an example scenario, if the contact quality between the user 401 and the reference electrode 414e and/or first electrode 414a is relatively poor (e.g., relatively small contact area between the user's skin and the surface of one or more of the electrodes 414a, 414e) the combined impedance may be relatively high. As a result, the amplitude of the first contact signal 408a may be relatively near, but less than, the amplitude of the reference signal 316. In another scenario, if contact is lost between the user 401 and the first electrode 414a (or reference electrode 414e), the combined impedance of the first electrode 414a and reference electrode 414e may become very high (e.g., the equivalent of an “open circuit”). The amplitude of the contact signal 408a may be very close (e.g., nearly equal) to the amplitude of the reference signal 316.

Using channel 4 as another example, in a scenario where the contact between the user 401 and the reference electrode 414e and fourth electrode 414d is relatively good (e.g., relatively large contact area between the skin of the user 401 and electrodes 414d-414e), the combined impedance may be relatively low. As a result, the fourth contact signal 408d has a lower amplitude, relative to the reference signal 316. As depicted in FIG. 4, the remaining electrodes 414b-414c may have an impedance such that when combined with the impedance of the reference electrode 414e, the second contact signal 408b may have a lower amplitude than the first contact signal 408a, and the third contact signal 408c may have an amplitude lower than the second contact signal 408b but greater than the fourth contact signal 408d. In other examples, the electrodes 414a-414e may have impedances that result in any of a variety of combinations of amplitudes of contact signals 408a-408d.

Each of the contact signals 408a-408d along with any acquired biological signals may be provided to the remainder of the processing circuitry 400 via a set of amplifiers 410a-410d, respectively. In some examples, the amplifiers 410a-410d may provide signal gain, which may be configurable. For instance, the amplifiers 410a-410d may each be configured to provide the same gain, or one or more of the amplifiers 410a-410d may be configured with different gains. In some variations, the gain may be dynamically controlled by the device, such as by a processor or other element. In further examples, one or more of the amplifiers 410a-d may provide unity gain (e.g., one or more of the amplifiers 410a-410d may be configured as a buffer). In still other examples, one or more of the amplifiers 410a-410d) may be configured to provide signal attenuation. In additional examples, the amplifiers 410a-410d may be configured to provide filtering. For instance, one or more of the amplifiers 410a-410d may include elements that provide active filtering of input signals (e.g., low-pass filtering, high-pass filtering, etc.).

The example processing circuitry 400 further includes a set of input switches 412 that sequentially connect the output of each of the amplifiers 410a-410d to an ADC 418, such as during a corresponding sampling window (e.g., sampling windows 302a-302d). For example, one of the input switches 412 may connect amplifier 410a (e.g., CH1) to the ADC 418 during the first sampling window 302a, while the remainder of the input switches 412 remain open (thereby disconnecting amplifiers 410b-410d from the ADC 418). During a subsequent sampling window (e.g., the second sampling window 302b), one of the input switches 412 may connect amplifier 410b (e.g., CH2) to the ADC 418, while the remainder of the amplifiers 410a, 410c, 410d remain disconnected. Such a process may continue through each of the channels on an ongoing basis during the measurement.

In examples, the input switches 412 may be implemented as any type of suitable switching element. For example, the input switches 412 may be implemented as a set of electro-mechanical switches, a set of semiconductor switches (e.g., a type of transistor), and/or any type of component suitable for connecting and disconnecting each amplifier 410a-410d from the ADC 418.

The ADC 418 may be any type of ADC suitable for digitizing the combined contact signals 408a-408d and biological signals acquired by the electrodes 414a-414d. For example, the ADC 418 may be a type of sigma-delta ADC, successive approximation ADC, or other type of ADC. Further, the ADC 418 may be configured to provide samples at a fixed sampling rate (which may be configurable). Based on the sampling rate, the ADC 418 may provide one or more data samples during each sampling window.

The processing circuitry 400 may also include a set of output switches 422 that connect the output of the ADC 418 to a set of filters 424a-424d. As described above for input switches 412, the output switches 422 may be implemented as any of a variety of suitable switching elements (e.g., electro-mechanical switches, transistors, etc.). In some examples, the output switches 422 may be controlled synchronously or near synchronously with the input switches 412. For instance, a common control signal may control the input switches 412 and output switches 422. In further examples, the input switches 412 and output switches 422 may be packaged together as a single element. In other examples, the input switches 412 and output switches 422 may be separate elements (as depicted) that are independently controlled.

Each channel of the processing circuitry 400 may further include a set of filters 424a-424d, which provide filtering of the samples output from the ADC 418. The filters 424a-424d may be any of a wide variety of filter types. In one example, the ADC 418 may be a sigma-delta ADC, which provides an output sampling rate that may be an order of magnitude (or more) higher than needed for analysis of the measured signals. In such examples, the filters 424a-424d may be a type of decimation filter that provides low-pass filtering of the ADC output. In other examples, the filters 424a-424d may be other types of filters or may otherwise provide other types of filtering.

The filtered outputs 426a-426d of each respective channel are provided for further processing, such as by a processor of the device (e.g., processor 102). The filtered outputs 426a-426d may each include a set of one or more data samples output from each respective filter 424a-424d during each channel's corresponding sampling window. In some examples each of the filtered outputs 426a-426d may be processed individually, while in other examples the filtered samples 426a-d may be processed in combination. For example, two or more of the filtered outputs 426a-426d may be subtracted from each other to yield a biological signal of interest (e.g., an ECG signal, and EMG signal, etc.). In further examples, processing of the filtered outputs 426a-426d may include additional filtering, processing by a type of algorithm (e.g., a denoising or other type of algorithm), and/or additional types of processing.

The filtered outputs 426a-426d are also provided to a first in, first out (FIFO) data buffer 428, where the samples may be stored. The output of the FIFO 428 is a composite signal 430 comprised of a sequential arrangement of samples from each of the sampling windows. The composite signal includes the combination of the contact signals 408a-408d, any biological signals acquired from the user 401, and/or any other signals acquired during each sampling window. The contact signals 408a-408d may be separated from the composite signal 430 and analyzed to determine a metric associated with electrode-skin contact.

As depicted in FIG. 4, one method for separating the sampled portions of the contact signals 408a-408d from the composite signal 430 is demodulation. For instance, the composite signal 430 may be input to a type of demodulator 432, such as an I/Q demodulator or other type of demodulator. A demodulation signal 434 may also be provided to the demodulator 432 and used in the demodulation process. In some examples, the demodulation signal 434 may be the reference signal 316, may be derived from the reference signal 316, or may be a representation of the reference signal 316 generated by another signal source (not depicted). In other examples, the demodulation signal 434 may be another signal associated with the reference signal 316, or may be another signal suitable for performing demodulation of the composite signal 430.

In some instances, the output of the demodulator 432 may be provided to an output filter 436, where demodulation artifacts and/or other unwanted signal components may be removed. The output filter 436 may be any of a variety of suitable filter types, such as a low-pass filter, high-pass filter, and/or other type of filter. In other instances the processing circuitry 400 may not include an output filter 436.

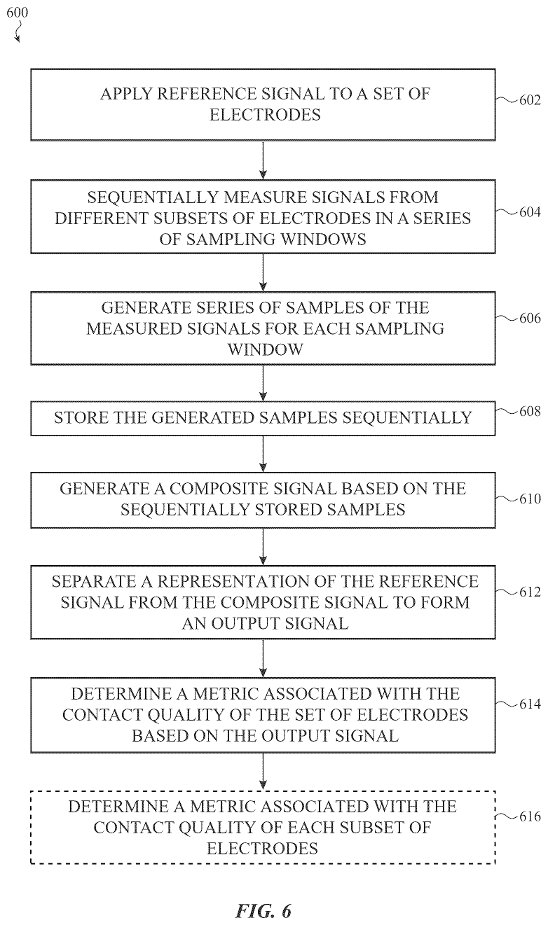

The output signal 438 of the output filter 436 may be comprised of samples that primarily represent portions of the contact signals 408a-408d sampled during the sampling window of each channel. Portions of the composite signal 430 representing biological and/or other signals may be significantly reduced or completely removed from the output signal 438 (such as resulting from the demodulation and/or filtering). The output signal 438 may be further analyzed to determine a metric associated with electrode-skin contact. Additional details are provided herein with respect to FIG. 6.

The example processing circuitry 400 depicted in FIG. 4 is one example of a variety of possible circuit topologies that may be used for performing electrode contact measurement. In other examples, other circuit topologies may also be suitable. For example, rather than including an amplifier 410a-410d for each electrode channel, the processing circuitry 400 may include a single amplifier that is switched between channels. Similarly, a single filter may be used at the output of the ADC 418 (in lieu of filters 424a-424d), and may be switched between each of the channel outputs in accordance with each sampling window. In other examples, in lieu of a single ADC 418, the processing circuitry 400 may include a plurality of ADCs, where a dedicated ADC may be provided for each electrode channel. In such an arrangement, switches 412, 422 may be excluded from the processing circuitry 400, or may be arranged differently than depicted in FIG. 4. In still other examples, processing circuitry 400 may be implemented via a range of other circuit topologies suitable for performing portions of the methods described herein.

As described herein, the output signal 438 provided by processing circuitry 400 represents sequential contributions from each of the sensing electrodes 414a-414d. In some instances, it may be desirable to be able to determine and analyze an output signal specific to a particular subset of electrodes (e.g., one or more of the electrode channels). FIG. 5 depicts portions of example of processing circuity 500 that may be included as part of a device and used to measure and process signals acquired from subsets of electrodes (e.g., electrode channels). Portions of the processing circuitry 500 may be configured in the same manner as processing circuitry 400, depicted in FIG. 4, with like components labeled the same. For example, processing circuitry 500 includes a signal generator 402, multiplexer 404, coupling capacitors 406a-406d, amplifiers 410a-410d, switches 412 and 422, ADC 418, filters 424a-424d, FIFO 428, demodulator 432, and output filter 436, which are configured to generate a demodulated and filtered output signal 438, as described herein with respect to FIG. 4. Processing circuitry 500 also includes components that generate four additional channel-specific outputs 538a-538d.

Each of the channel-specific output signals 538a-538d is generated by further processing each of the corresponding filtered outputs 426a-426d, which are input to a set of demodulators 532a-532d, respectively. Each of the demodulators 532a-532d may be any type of demodulator suitable for separating portions of each of the contact signals 408a-408d from biological signals and/or other signals that may be present in each of the filtered outputs 426a-426d. For instance, the demodulators 532a-532d may be an I/Q demodulator or other type of demodulator. In some examples, the demodulators 532a-532d may be the same type of demodulator as demodulator 432.

As described herein for demodulator 432, each of the demodulators 532a-532d also receives a demodulation signal 534a-534d, respectively, which are used to perform the demodulation. In some variations, the demodulation signals 534a-534d may all be the same signal. For example, the demodulation signals 534a-534d may be the reference signal 316, may be derived from the reference signal 316, or may be a representation of the reference signal 316 generated by another signal source (not depicted). In other examples, the demodulation signal 534a-534d may be another signal associated with the reference signal 316, or may be another signal suitable for performing demodulation. Further, the demodulation signals 534a-534d may the same as demodulation signal 434. In other variations, one or more of the demodulation signals 534a-534d may be different from the others and/or may be different from demodulation signal 434.

The output signal from each demodulator 532a-532d is provided to an output filter 536a-536d, respectively. Each of the output filters 536a-536d may be any type of filter suitable for providing channel-specific output signals 538a-538d for further processing. For example, each of the filters 536a-536d may be any of a low-pass filter, high-pass filter, band-pass filter, and/or any other type of suitable filter. The output filters 536a-536d may each have a different cutoff frequency, pass band, roll-off, and/or may have other filter parameters that are unique to each of the output filters 536a-536d. In some example, the output filters 536a-536d may be the same type of filter with the same filter parameters. In further examples, the output filters 536a-536d may or not be the same as output filter 436.

As described herein with respect to output signal 438, the channel-specific output signals 538a-538d, may be comprised of samples that primarily represent portions of the contact signals 408a-408d, respectively, sampled during the corresponding sampling window of each channel. Portions of each output signal 426a-426d representing biological and/or other signals may be significantly reduced or completely removed from the channel-specific output signals 538a-538d (such as resulting from the demodulation and/or filtering). Each of the channel-specific output signals 538a-538d may then be further analyzed and/or processed to determine a metric associated with the quality of contact between each individual electrode 414a-414d and the user 401. Additional details are provided herein with respect to FIG. 6.

FIG. 6 depicts an example method 600 for determining a metric associated with electrode-skin contact. Method 600 may be performed during a measurement or following the measurement, as described herein. The steps of method 600 may be stored as instructions on a non-transitory computer-readable storage device (e.g., memory 104), such that one or more processors (e.g., processor(s) 102) operatively coupled to the memory may utilize these instructions to perform the various steps of the processes described herein. The electrodes, processor(s), memory, and/or other elements associated with method 600 may be part of an electronic device (e.g., device 100, 200).

At operation 602, a reference signal is applied to a set of electrodes (e.g., electrodes 414a-414d). The reference signal may be any of a range of signals suitable for determining electrode-skin contact. The reference signal may be configured in any manner as described herein with respect to the reference signal 316 of FIGS. 3A and 3B. For example, in some variations the reference signal may be a sinusoidal signal having a reference frequency (e.g., Fmod). In instances where the devices 400 and 500 of FIGS. 4 and 5 are used to perform the method 600, this may include generating the reference signal using signal generator 402.

In some examples, the reference signal may be applied simultaneously to the set of electrodes. The set of electrodes may include one or more subsets of electrodes, where each subset may include two or more electrodes associated with an electrode channel. In other examples, the reference signal may be applied sequentially to each electrode or subset of electrodes (e.g., to each electrode channel), depending on which subset of electrodes is currently being measured.

The reference signal may be applied to each electrode or subset of electrodes via a set of coupling capacitors (e.g., capacitors 406a-406d). The impedance of each coupling capacitor, in combination with the impedance of one or more of the electrodes, may result in attenuation of the coupled reference signal. Thus, a contact signal appears at each electrode (e.g., contact signals 408a-d) as part of an input signal to the device processing circuitry.

At operation 604, signals are sequentially measured from different subsets of electrodes sequentially in a series of sampling windows (e.g., sampling windows 302a-302d), as described herein. The measured signals for each subset of electrodes may include a combination of a contact signal (e.g., an attenuated representation of the reference signal), biological signal(s), noise and/or measurement artifact, and/or other signals. The measurement may include applying a gain to the measured signals (e.g., via amplifiers 410a-410d), filtering the signals (e.g., via filters 424a-424d), and/or performing other measurement operations. The measured signals may be acquired from each subset of electrodes via a set of one or more switches (e.g., switches 412, 422) that sequentially switch each electrode channel during each corresponding sampling window.

At operation 606, a series of one or more samples (e.g., samples 318a, 320a, etc.) is generated for each of the measured signals during each corresponding sampling window. For example, the processing circuitry may include an ADC (e.g., ADC 418). During each sampling widow, a switch may provide connection of the measured signal acquired from each subset of electrodes to the ADC, which may generate a series of one or more samples of the measured signal.

At operation 608, the samples generated for each electrode channel during the sampling window are stored sequentially. For example, the samples may be stored in a FIFO (e.g., FIFO 428) or other type of memory element. At operation 610, a composite signal (e.g., composite signal 430) is generated based on the sequentially stored samples. The composite signal is comprised of segments which include the measured signals from each of the subsets of electrodes.

At operation 612, a representation of the reference signal is separated from the composite signal to form an output signal (e.g., output signal 438). The output signal may be comprised substantially of the representation of the reference signal, where the biological and/or other signals present in the measured signals may be removed from the output signal. The representation of the reference signal may be separated from the composite signal by any of a variety of suitable methods, as described herein, such as by a type of demodulation (e.g., I/Q demodulation).

At operation 614, a metric associated with the contact quality of the set of electrodes is determined based on the output signal. In one example, the determined metric may be the average value of the output signal. For instance, the average may be a rolling average, determined over a number of samples, determined over one or more periods of the output signal, or otherwise determined from the output signal. In another example, the determined metric may be associated with the root mean square (RMS) of the output signal, determined over a time interval of the output signal. As yet another example, the determined metric may be the area under the curve of the output signal, such as may be determined by mathematical integration performed over a number of samples or a time interval. In still other examples, the determined metric may be any of a wide variety of suitable metrics that may be associated with electrode-skin contact quality.

In some variations, determining the metric may include performing additional operations on the output signal. For example, an absolute value of the output signal may be determined, or the output signal may otherwise be rectified, to eliminate negative amplitude values. The metric may then be determined accordingly.

In some examples, the device may modify an aspect of the measurement based on the value of the determined metric. For instance, in examples where the metric is an average value of the output signal, the average value may be compared to a predetermined threshold or a range of allowable values. If the metric exceeds the threshold or is outside the range of allowable values, the device may determine that the contact quality between the skin and the electrode does not support a desired level of signal quality. Based on this determination, the device may modify an aspect of the measurement. For instance, in examples where the device may perform method 600 during a measurement, the device may terminate the measurement. In other examples, based on the value of the determined metric, the device may disregard a plurality of data samples, such as during one or more periods of the measurement when the contact quality is poor. The device may notify the user accordingly. In examples where the device may perform method 600 following the completion of a measurement, the device may disregard a plurality of samples acquired during intervals of poor contact quality, or may disregard the entire measurement.

At operation 616, a metric associated with the contact quality of each subset of electrodes may optionally be determined. For example, the device may include processing circuitry that provides one or more channel-specific output signals (e.g., channel-specific output signals 538a-538d). Each channel-specific output signal may include a representation of the reference signal specific to a subset of electrodes (e.g., an electrode channel), acquired during a corresponding sampling window. Thus, each channel-specific output signal includes samples that reflect the contact quality of each of the subset of electrodes, rather than all of the electrodes.

As described for operation 614, the metric associated with each subset of electrodes may be a type of signal average, an RMS value, an area under the curve, and/or another type of metric. The metric determined for each channel-specific output signal may be used as described herein to modify an aspect of the measurement.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining a metric, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, the output result may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method, comprising:
applying a reference signal to a set of electrodes;
sequentially measuring, during a series of sampling windows, different subsets of the set of electrodes to generate a series of samples for each of the sampling windows;
sequentially storing, for each of the series of sampling windows, the corresponding series of samples;
generating, based on the sequentially stored series of samples for each of the sampling windows, a composite signal;
separating a representation of the reference signal from the composite signal to form an output signal; and
determining, based on the output signal, a metric associated with contact quality of the set of electrodes.

2. The method of claim 1, wherein separating the representation of the reference signal from the composite signal comprises performing demodulation of the composite signal.

3. The method of claim 2, wherein performing the demodulation comprises performing I/Q demodulation.

4. The method of claim 2, wherein the demodulation is performed using the reference signal as a demodulation signal.

5. The method of claim 2, wherein the reference signal is applied simultaneously to each subset of the set of electrodes.

6. The method of claim 2, wherein the reference signal is applied to the subsets of the set of electrodes through a set of coupling capacitors.

7. The method of claim 2, wherein the reference signal is a sinusoid having a reference frequency.

8. The method of claim 7, wherein the series of samples generated for each of the sampling windows is generated at a measurement frequency that is an integer multiple of the reference frequency.

9. The method of claim 8, wherein the measurement frequency is a multiple of two of the reference frequency.

10. A device comprising:
a set of electrodes;
a signal generator configured to apply a first signal having a first frequency to each of a plurality of subsets of the set of electrodes; and
processing circuitry configured to:
sequentially measure each subset of the plurality of subsets of the set of electrodes to obtain a plurality of acquired signals, wherein each of the plurality of the acquired signals includes the first signal and a corresponding biological signal measured by the corresponding subset of the plurality of subsets of the set of electrodes;
digitize the plurality of acquired signals;
generate a composite signal using the digitized plurality of acquired signals;
demodulate the composite signal to separate the first signal from the corresponding biological signals of the digitized plurality of acquired signals; and
determine a metric associated with a contact quality of the set of electrodes using the demodulated composite signal.

11. The device of claim 10, wherein demodulating the composite signal comprises using I/Q demodulation.

12. The device of claim 10, wherein the metric associated with contact quality is an average of the demodulated composite signal.

13. The device of claim 10, further comprising a set of coupling capacitors, wherein the first signal is coupled by the set of coupling capacitors to each of the plurality of subsets of the set of electrodes.

14. The device of claim 10, wherein the first signal is a sinusoid.

15. The device of claim 14, wherein the plurality of subsets of the set of electrodes is measured at a measurement frequency that is an integer multiple of the reference frequency.

16. The method of claim 15, wherein: wherein the measurement frequency is a multiple of two of the first frequency.

17. A device comprising:
a set of electrodes;
a signal generator configured to simultaneously apply a reference signal to each of a plurality of subsets of the set of electrodes; and
processing circuitry configured to:
sequentially measure, during a series of sampling windows, each of the plurality of subsets of the set of electrodes to generate a series of samples for each of the corresponding sampling windows in the series of sampling windows;
sequentially store, for each of the series of sampling windows, the corresponding series of samples;
generate, based on the sequentially stored series of samples for each of the sampling windows, a composite signal;
separate a representation of the reference signal from the composite signal to form an output signal; and
determine, based on the output signal, a metric associated with contact quality of the set of electrodes.

18. The device of claim 17, wherein separating the representation of the reference signal from the composite signal comprises performing demodulation of the composite signal.

19. The device of claim 17, wherein the metric associated with contact quality is an average of the output signal determined by the device.

20. The device of claim 17, wherein the processing circuitry is further configured to:
generate, for each of the plurality of subsets of the set of electrodes, a corresponding output signal;
wherein each corresponding output signal represents a portion of a contact signal acquired during a corresponding sampling window for each of the plurality of subset of the set of electrodes; and
wherein each corresponding output signal is formed by separating the portion of the contact signal from a plurality of acquired signals that includes at least the contact signal and a biological signal.
